(19) United States
(12) Patent Application Publication
(10) Pub. No.: US 2015/0335558 A1
(43) Pub. Date: Nov. 26, 2015

(54) NERVE GROWTH INHIBITOR, CUTANEOUS-SENSORY-IRRITATION INHIBITOR, AND MARKER FOR CUTANEOUS-SENSORY-IRRITATION DETECTION

(71) Applicant: HOYU CO., LTD., Nagoya-shi, Aichi (JP)

(72) Inventor: Yoshihiro Inami, Aichi (JP)

(21) Appl. No.: 14/437,425

(22) PCT Filed: Oct. 17, 2013
(86) PCT No.: PCT/JP2013/078223

§ 371 Date: Aug. 11, 2015

(30) Foreign Application Priority Data
Oct. 22, 2012 (JP) 2012-232634

(51) Int. Cl. A61K 8/49; A61K 31/7034; A61K 8/34; A61K 31/05; A61K 31/352; A61K 8/37; A61K 31/235; A61K 31/7048; A61K 8/97; A61K 36/52; A61K 36/736; A61K 36/69; A61K 36/07; A61K 36/53; A61K 36/284; G01N 33/573; A61K 8/60

(52) U.S. Cl. CPC A61K 8/498; A61K 8/602; A61K 31/7034; A61K 8/347; A61K 31/05; A61K 31/352; A61K 8/375; A61K 31/235; A61K 31/7048; A61K 8/97; A61K 36/52; A61K 36/736; A61K 36/69; A61K 8/975; A61K 36/07; A61K 36/53; A61K 36/284; G01N 33/573; G01N 2333/988; G01N 2500/04; G01N 2500/10

(57) ABSTRACT

Provided are a nerve growth inhibitor and a cutaneous-sensory-irritation inhibitor, with which dermal hyperesthesia and nerve extension based on newly found nerve extension mechanisms can be effectively inhibited. This nerve growth inhibitor or cutaneous-sensory-irritation inhibitor comprises one or more selected from: (1) a specific flavonoid; (2) a tannin; (3) chlorogenic acid; (4) a specific stilbenoid; and a glycoside or a pharmaceutically acceptable salt of (1)-(4); or (5) a specific crude drug extract; and (6) a walnut polyphenol.

### TECHNICAL FIELD

The present invention relates to a nerve growth inhibitor, a cutaneous-sensory-irritation inhibitor and a marker for cutaneous-sensory-irritation. More specifically, the present invention relates to a nerve extension inhibitor that inhibits nerve extension based on a newly discovered extension mechanism of the free nerve ending (referred to hereinafter simply as the “nerve”) of the skin, a dermal hyperesthesia inhibitor that inhibits dermal hyperesthesia as a result of inhibiting nerve extension, compositions containing these inhibitors, and a dermal hyperesthesia detection marker for detecting or predicting dermal hyperesthesia based on the above nerve extension mechanism.

### BACKGROUND ART

The human skin or mammalian skin is composed of epidermis, dermis and hypodermis. The free nerve ending, which is a peripheral sensory nerve, normally reaches a close proximity of the boundary between the dermis and the epidermis, but it does not extend into the epidermis. However, skins with hyperesthesia, such as the so-called sensitive skin, dry skin, or rough skin, have nerve endings that extend into the epidermis. Moisturizers are used for such sensitive skin, etc. to block external stimuli from acting on nerves extending into the epidermis, but no satisfactory effect has been obtained to date.

In view of such reports as Non-Patent Document 1 shown below, which indicate that the above nerve extension causes itches and other unpleasant dermal hyperesthesia, it is more natural to think that a fundamental and effective solution against dermal hyperesthesia is to inhibit the very act of the nerve extending into the epidermis or to make the nerve extending into the epidermis shrink and withdraw to a normal state.

The nerve growth factor (NGF) and the nerve extension inhibiting factor (Sema3A: Semaphorin 3A) are known in relation to such nerve extension, and disclosures exist of a nerve extension inhibitor and a dermal hyperesthesia inhibitor using substances that affect the above factors. For example, Patent Document 1 mentioned below proposes a nerve extension inhibitor and an antipruritic drug containing a certain crude drug based on the idea that the action of histamine on keratinocytes in the epidermis accelerates NGF generation in the keratinocytes and results in nerve extension into the epidermis.

The ideas in the above published documents, such as Non-Patent Document 1 and Patent Document 1, that is, the ideas that nerve extension causes dermal hyperesthesia, that nerve extension is caused by NGF generation in keratinocytes, and that the action of histamine causes NGF generation in keratinocytes, are almost completely accepted by many experts.

However, when considering the above histamine that causes nerve extension, the dominant view is that “the mast cells existing in the dermis of the skin store histamine generated by L-histidine decarboxylase (HDC), and degranulation of the mast cells releases histamine from cells, so that it acts on keratinocytes as described above” as suggested directly or indirectly in the following Patent Documents 2-5.

### CITATION LIST

### Non-Patent Documents

Non-Patent Document 1: Tobin D., Nabaro G. de la Faille, H. B. van Vloten, W. A., van derPutte, S. C. J., Schuurman, H.-J., 1992 “Increased number of immunoreactive nerve fibers in atopic dermatitis” J. Allergy Clin. Immunol. 90, 613-622

### PATENT DOCUMENTS

Patent Document 1: JP Publication No. 2010-1264

Patent Document 2: JP Publication No. H08-217674

Patent Document 3: JP Publication No. H09-110857

Patent Document 4: JP Publication No. H10-059956

Patent Document 5: JP Publication No. 2006-176480

### SUMMARY

### Technical Problem

As shown above, a conventional nerve extension inhibitor is created by assuming that histamine, which causes nerve extension, is derived from the mast cell existing in the dermis of the skin.

However, a study by the present inventors led to a result which indicates that even a model mast cell deficient mouse may experience nerve extension, which is not easily predictable from the conventional common technical knowledge. This result suggests a possibility that nerve extension may be caused by histamine derived from a new unknown origin. It can thus be presumed that a conventional nerve extension inhibitor or a dermal hyperesthesia inhibitor that is adapted to histamine derived from mast cells in the dermis may not be effective in inhibiting nerve extension caused by histamine of the new unknown origin.

Meanwhile, it is well known that histamine generated from L-histidine by the activity of HDC relates to the itch in the skin, and that mast cells in the dermis contain active HDC and store histamine. In addition to the conventional view that histamine released from mast cells by degranulation of the cells “acts on keratinocyte to accelerate NGF generation,” the view that this histamine “binds to the histamine receptor existing on the sensory nerve and sends an itch signal to the sensorium” is also dominant.

However, the study of the present inventors in relation to the above itch uncovered the presence of a new type of itch that cannot be explained by the mechanism of an itch based on histamine released from mast cells by degranulation. This point is described in detail in Japanese patent application No. 2011-265760 (Prior Application 1), previously filed by the present applicant, so only a brief overview will be provided in this document. More specifically, an itch caused by a new itch developing mechanism described below has been uncovered. “The keratinocytes in the epidermis of the skin contain inactive HDCs (HDC precursors) that are activated by the action of a stimulating substance. As a result, histamine is generated in the keratinocytes and released from the cells. This makes the skin itch.”

In addition, the present applicant has previously filed Japanese patent application No. 2012-074733 (Prior Application 2). In this Prior Application 2, the applicant specifically disclosed a new and effective HDC activation inhibitor, which was discovered based on the knowledge of Prior Application 1. The “HDC activation inhibitor” is a substance that inhibits the inactive HDC in keratinocytes from being activated by the action of a stimulating substance, and thus, prevents generation of histamine in the keratinocytes.

The present inventors considered the relationship of a new finding that “even a model mast cell deficient mouse may experience nerve extension” and a new finding that “when the inactive HDCs in the keratinocytes are activated by the action of a stimulating substance, histamine is generated in the keratinocytes,” and arrived at a hypothesis that the “histamine of a new unknown origin” is a “histamine that develops in the keratinocytes by activation of inactive HDC.”

If this hypothesis is true, the histamine relating to nerve extension would not be histamine derived from mast cells. In other words, the desired inhibitory effect will not be obtained by the conventional practice of “inhibiting degranulation of the mast cells” to inhibit nerve extension into the epidermis. That is to say, the nerve extension inhibitor based on conventional knowledge would not be able to inhibit the newly found source of histamine, namely “activation of HDC in the keratinocytes,” per se, and thus, it will not have the desired effect against nerve extension.

Accordingly, the present invention is directed to providing a nerve extension inhibitor and a dermal hyperesthesia inhibitor that can inhibit nerve extension by histamine of a new, unknown origin, and to provide a dermal hyperesthesia detection marker to detect or predict dermal hyperesthesia by such nerve extension mechanism as a technical problem to be solved.

The present inventors conducted further experiments using the HDC activation inhibitor specifically disclosed in Prior Application 2 to verify that the above hypothesis, that is, the hypothesis that “histamine of a new unknown origin” is a “histamine that develops in the keratinocytes by activation of inactive HDC,” is true, and completed the present invention.

### Solution to Problem

[Arrangement of Invention 1]

The arrangement of Invention 1 to solve the above problem is a nerve extension inhibitor that is one or more types selected from (1) to (6) below:

(1) Apigenin, luteolin, diosmetin, genkwanin, poncirin, eriodictyol, sterubin, prunetin, which belong to flavonoids, or glycosides or pharmaceutically acceptable derivatives thereof;

(2) Tannin or glycosides or pharmaceutically acceptable derivatives thereof;

(3) A chlorogenic acid or glycosides or pharmaceutically acceptable derivatives thereof;

(4) Stilbenoids encompassing at least resveratrol or glycosides or pharmaceutically acceptable derivatives thereof;

(5) A Prunus jamasakura bark extract, a Polygala root extract, a Hoelen extract, a Glechoma hederacea extract, or an Atractylodis lanceae rhizoma extract, which are crude drug extracts; and

(6) Walnut polyphenol.

[Arrangement of Invention 2]

The arrangement of Invention 2 for solving the above problem is a nerve extension inhibitor composition containing the nerve extension inhibitor according to Invention 1.

[Arrangement of Invention 3]

The arrangement of Invention 3 for solving the above problem is the nerve extension inhibitor composition according to Invention 2, wherein the nerve extension inhibitor composition is a drug, a quasi-drug or cosmetics.

[Arrangement of Invention 4]

The arrangement of Invention 4 for solving the above problem is the nerve extension inhibitor composition according to either Invention 2 or Invention 3, wherein the nerve extension inhibitor composition is an external preparation for skin.

[Arrangement of Invention 5]

The arrangement of Invention 5 for solving the above problem is a dermal hyperesthesia inhibitor that is one or more types selected from (1) to (6) below:

(1) Apigenin, luteolin, diosmetin, genkwanin, poncirin, eriodictyol, sterubin, prunetin, which belong to flavonoids, or glycosides or pharmaceutically acceptable derivatives thereof;

(2) Tannin or glycosides or pharmaceutically acceptable derivatives thereof;

(3) A chlorogenic acid or glycosides or pharmaceutically acceptable derivatives thereof;

(4) Stilbenoids encompassing at least resveratrol or glycosides or pharmaceutically acceptable derivatives thereof;

(5) A Prunus jamasakura bark extract, a Polygala root extract, a Hoelen extract, a Glechoma hederacea extract, or an Atractylodis lanceae rhizoma extract, which are crude drug extracts; and

(6) Walnut polyphenol.

[Arrangement of Invention 6]

The arrangement of Invention 6 for solving the above problem is a dermal hyperesthesia inhibitor composition containing a dermal hyperesthesia inhibitor according to Invention 5.

[Arrangement of Invention 7]

The arrangement of Invention 7 for solving the above problem is the dermal hyperesthesia inhibitor composition according to Invention 6, wherein the dermal hyperesthesia inhibitor composition is a drug, a quasi-drug or cosmetics.

[Arrangement of Invention 8]

The arrangement of Invention 8 for solving the above problem is the dermal hyperesthesia inhibitor composition according to either Invention 6 or Invention 7, wherein the dermal hyperesthesia inhibitor composition is an external preparation for skin.

[Arrangement of the Invention 9]

The arrangement of Invention 9 for solving the above problem is a dermal hyperesthesia detection marker that is at least one of an active HDC or an inactive HDC in a skin keratinocyte sample.

### ADVANTAGEOUS EFFECTS OF INVENTION

The nerve extension inhibitors listed as (1) to (6) in Invention 1 work in animals, particularly mammals encompassing human and non-human mammals, to effectively prevent or inhibit, a state of the so-called sensitive skin, dry skin, or rough skin, in which the free nerve ending, which is a peripheral sensory nerve of the skin, extends into the epidermis. This is possible because these nerve extension inhibitors inhibit histamine generation based on activation of inactive HDC in the keratinocytes, and thus, prevent the free nerve ending from extending into the epidermis or make the free nerve ending that has extended into the epidermis shrink and withdraw to the dermis.

The dermal hyperesthesia inhibitors listed as (1) to (6) in Invention 5 effectively prevent the free nerve ending from extending into the epidermis, for the same reason as Invention 1, and thus, effectively prevent or inhibit dermal hyperesthesia, such as the so-called sensitive skin, dry skin, or rough skin.

The nerve extension inhibitor composition of Invention 2 provides a nerve extension inhibitor composition that can effectively inhibit the new nerve extension mechanism discovered by the present inventors, since it contains the nerve extension inhibitor according to Invention 1. Also, the dermal hyperesthesia inhibitor composition of Invention 6 provides a dermal hyperesthesia inhibitor composition that can effectively inhibit dermal hyperesthesia, such as sensitive skin, dry skin, or rough skin, based on the new nerve extension mechanism discovered by the present inventors, since it contains the dermal hyperesthesia inhibitor of Invention 5.

As shown in Invention 3 or Invention 7, the nerve extension inhibitor composition or the dermal hyperesthesia inhibitor composition can be used as drugs, quasi-drugs or cosmetics.

As shown in Invention 4 or Invention 8, the nerve extension inhibitor composition or the dermal hyperesthesia inhibitor composition can preferably be used in particular as external preparation for skin to inhibit sensitive skin, dry skin, or rough skin.

As mentioned above, when the inactive HDC (HDC precursor) existing in keratinocytes in the epidermis of the skin is activated by a stimulating substance, histamine is generated in keratinocytes and released outwardly. The released histamine causes nerve extension, which leads to dermal hyperesthesia. Accordingly, as shown in Invention 9, at least one of active HDC and inactive HDC in the skin keratinocyte sample can be used as a marker for detecting dermal hyperesthesia. In other words, dermal hyperesthesia can be predicted or diagnosed based on the measured amount of inactive HDC in a specific skin keratinocyte sample (A) relative to the standard amount of inactive HDC existing in keratinocytes in the epidermis of the skin. Furthermore, dermal hyperesthesia can be similarly predicted or diagnosed based on the measured amount of active HDC (B) or based on the ratio of (A) and (B).

### BRIEF DESCRIPTION OF DRAWINGS

FIG. 1 shows the analysis result of Sample Experiment 1-1.

FIG. 2 shows the analysis result of Sample Experiment 1-2.

FIG. 3 shows the analysis result of Sample Experiment 1-3.

FIG. 4 shows the result of analyzing the temporal change of scratch bouts by a single local application of a 10% sodium laurate (SL) aqueous solution by using mast cell deficient mice and wild-type mice. The graphs are shown by an average amount±standard error (n=7).

FIG. 5 shows the result of analyzing the histamine content and the amount of HDC expression in the epidermis after a single local application of a 10% sodium laurate (SL) aqueous solution. The graphs are shown by an average amount±standard error (n=3). Note that FIG. 5(b) is a result of Western blot analysis of the amount of HDC expression in the epidermis, and FIG. 5(c) shows the values of HDC expression amount/β-actin expression amount based on the result of the Western blot analysis of FIG. 5 (b).

FIG. 6 shows a result of analyzing the histamine content and the amount of HDC expression in the dermis 2 hrs. after a single local application of a 10% sodium laurate (SL) aqueous solution. The graphs are shown by an average amount±standard error (n=3). Note that FIG. 6(b) is a result of Western blot analysis of the amount of HDC expression in the dermis, and FIG. 6(c) is the values of HDC expression amount/β-actin expression amount based on the result of the Western blot analysis of FIG. 6(b).

FIG. 7 shows an example of an image of a section from a skin sample by a fluorescence phase-contrast microscope.

FIG. 8 shows a graph of signal strength of the Examples by an image analysis software.

FIG. 9 shows a graph of signal strength of the Examples by an image analysis software.

FIG. 10 shows a graph of signal strength of the Examples by an image analysis software.

FIG. 11 shows a graph of signal strength of the Examples by an image analysis software.

### DESCRIPTION OF EMBODIMENTS

Next, embodiments of the present invention including the best mode thereof are explained below.

[Nerve Extension Inhibitor, Dermal Hyperesthesia Inhibitor]

The nerve extension inhibitor or dermal hyperesthesia inhibitor is broadly interpreted as a nerve extension inhibitor that is one or more types selected from (1) to (6) below:

(1) Flavonoids, or glycosides or pharmaceutically acceptable derivatives thereof;

(2) Tannin or glycosides or pharmaceutically acceptable derivatives thereof;

(3) A chlorogenic acid or glycosides or pharmaceutically acceptable derivatives thereof;

(4) Stilbenoids encompassing at least resveratrol or glycosides or pharmaceutically acceptable derivatives thereof;

(5) Crude drug extracts;

(6) Walnut polyphenol.

<Flavonoid>

The above-mentioned flavonoid is a type of polyphenol, and it is normally a collective name of a plant secondary metabolite originating in chalcone, which is composed by polymerization of coumarate-CoA and malonyl-CoA. Flavonoids encompass anthocyanin, flavan, flavanone, flavone, flavonol, dihydroflavonol, chalcone, aurone, isoflavonoid and neoflavonoid. A plant extract containing these substances includes a yerba santa leave extract.

The nerve extension inhibitor or dermal hyperesthesia inhibitor of the present invention is more specifically a nerve extension inhibitor or dermal hyperesthesia inhibitor composed of one or more of (1) to (6) below. We have not heard any reports of them as a nerve extension inhibitor or dermal hyperesthesia inhibitor of the present invention, nor any reports of them as a conventional nerve extension inhibitor or dermal hyperesthesia inhibitor.

(1) Apigenin, luteolin, diosmetin, genkwanin, poncirin, eriodictyol, sterubin, prunetin, which belong to flavonoids, or glycosides or pharmaceutically acceptable derivatives thereof;

(2) Tannin or glycosides or pharmaceutically acceptable derivatives thereof;

(3) A chlorogenic acid or glycosides or pharmaceutically acceptable derivatives thereof;

(4) Stilbenoids encompassing at least resveratrol or glycosides or pharmaceutically acceptable derivatives thereof;

(5) A Prunus jamasakura bark extract, a Polygala root extract, a Hoelen extract, a Glechoma hederacea extract, or an Atractylodis lanceae rhizoma extract, which are crude drug extracts; and

(6) Walnut polyphenol.

In the above, a “glycoside” is a compound in which a functional group, such as a hydroxyl group or a carboxyl group, of any of (1) to (4) above binds to a single or multiple sugar units, such as glucose, galactose, or the like, as far as they do not inhibit the nerve extension inhibition effect or the dermal hyperesthesia inhibition effect.

Further, a “pharmaceutically acceptable derivative” is a compound of any of (1) to (4) above in which a functional group, such as a hydroxyl group or a carboxyl group, binds to any other compound, or in which a specific carbon atom constituting the ring structure is substituted with any other compound, as far as they are pharmaceutically acceptable. Pharmaceutically acceptable derivatives include, for example, various salts, solvates, or esterified compounds.

Examples of salts include inorganic acid salts (e.g. hydrochloric acid salts, sulfuric acid salts, nitric acid salts, hydrobromic acid salts, phosphoric acid salts), organic acid salts (e.g. carboxylic acid salts, oxycarboxylic acid salts, organic sulfonic acid salts), salts with organic base (e.g. methylamine, triethylamine, triethanolamine) and salts with inorganic base (e.g. ammonium salts, alkali metal salts, alkali earth metal salts). Examples of solvates include hydrates, ethanol solvates, methanol solvates, acetonitrile solvates. Examples of esterified compounds include carboxylic acid esters, phosphoric acid esters, carbonic acid esters, sulfuric acid esters, nitric acid esters, and thioesters.

Derivatives also encompass the so-called prodrugs. Prodrugs are metabolic precursors that can be transformed into the compound of the present invention under a physiological condition.

<Tannin>

Tannin in the present invention is the bitterness component of a persimmon juice or an astringent skin of a chestnut, and it is a collective name of polyphenol compounds contained in various other plant leaves as well. A typical tannin includes plant tannin from Rhus javanica or gallnuts, persimmon tannin, chestnut skin tannin, tamarind tannin, mimosa tannin, and tannins included therein. Tannin of the present invention also includes hydrolytic tannins (pyrogallol tannin) and condensed tannins (catechol tannin). Specific examples of tannin include tannic acid, and hydrolytic tannin included in clove, and tannic acid is more preferable.

<Chlorogenic Acid>

Chlorogenic acid is also called 5-caffeoylquinic acid, and it is formed by a dehydration/condensation reaction of the carboxyl group of the caffeic acid and a hydroxyl group at position 5 of the quinic acid. It can be obtained from coffee beans and seeds and leaves of many other types of dicotyledons. To give an example of a dicotyledon, it is included in cherry leaves.

<Stilbenoid>

Stilbenoid is a compound composed of p-hydroxycinnamic acid CoA binding with 3 units of malonyl-CoA and forming a cyclized structure. Examples of stilbenoid include various stilbenes encompassing resveratrol, rhaponticin, as well as phyllodulcin, oligostilbene, polystilbene; other examples include oligomers of resveratrol, such as ε-viniferin, gnetin C, which are resveratrol dimers, or gnemonoside A, gnemonoside C and gnemonoside D, which are glycosides of a resveratrol dimer, or α-viniferin, which is a resveratrol trimer, or vaticanol C, which is a resveratrol tetramer. A preferred resveratrol is the type having a systematic name of 3,5,4′-trihydroxy-trans-stilbene.

<Walnut Polyphenol>

A walnut polyphenol is a component contained in the seed coat (thin skin), and it is a hydrolytic polyphenol.

<Crude Drug Extract>

The Prunus jamasakura bark extract, Polygala root extract, Hoelen root extract, Glechoma hederacea extract, and Atractylodis lanceae rhizoma extract are respectively crude drug extracts extracted from the crude drugs, Prunus jamasakura bark, Polygala root, Hoelen root, Glechoma hederacea, and Atractylodis lanceae rhizoma.

[Nerve Extension Inhibitor Composition, Dermal Hyperesthesia Inhibitor Composition]

<Active Components, Usages and Formulations>

The nerve extension inhibitor composition or dermal hyperesthesia inhibitor composition of the present invention contains the above nerve extension inhibitors or the above dermal hyperesthesia inhibitors as the active component. The content of the nerve extension inhibitor in the nerve extension inhibitor composition or the content of the dermal hyperesthesia inhibitor in the dermal hyperesthesia inhibitor composition is not limited, but the lower limit of these contents may be set to 0.1 mass %, for example. A content that is lower than 0.1 mass % may not provide a satisfactory effect. In addition, the upper limit of these contents may be kept at about 2 to 5 mass %, and 10 mass % at the highest, considering the balance between the effect and the cost. However, the content may be increased to about 50 mass % as necessary. A content that exceeds 50 mass % may result in problems of solubility, etc.

The nerve extension inhibitor composition or dermal hyperesthesia inhibitor composition of the present invention may be used as a drug, a quasi-drug or cosmetics having various usages to treat and/or prevent various symptoms that is accompanied by hypersensitivity of the skin or an itchy feeling of the skin. A particularly preferable example is an external preparation for skin, but an oral medicine, injections and the like are also preferably used.

Usages of an external preparation for the skin include drugs, quasi-drugs or cosmetics for treating, reducing or stopping the progression of symptoms of, or preventing dermal hyperesthesia, dry-type dermal pruritus, seborrheic dermal pruritus, sensitive skin, dermal pruritus involving inflammation. Examples of typical dermal diseases are xeroderma, atopic dermatitis, psoriasis and contact dermatitis.

The nerve extension inhibitor composition or dermal hyperesthesia inhibitor composition of the present invention can be prepared in various formulations. For example, it can be prepared as a solid medicine including that in a stick shape, an ointment, a liquid drug including lotion, emulsion or aerosol, a foam, a gel, a cream, a patch including packs, etc. In particular, an ointment, a liquid drug, a gel and a cream are preferable.

Methods for preparing drugs in various formulations are not particularly limited, and the drugs may be prepared by a common method by appropriately selecting and blending various components. In addition, the dosage or method of using the nerve extension inhibitor composition or dermal hyperesthesia inhibitor composition of the present invention is not particularly limited, and normally, an appropriate amount can be applied a few times a day.

<Other Components>

The nerve extension inhibitor composition or dermal hyperesthesia inhibitor composition of the present invention can contain at least one known antipruritic agent, nerve extension inhibitor or dermal hyperesthesia inhibitor, as long as it does not inhibit the effect of the present invention.

Known antipruritic agents include for example chlorpheniramine, diphenhydramine, lidocaine, dibucaine, aminobenzoic acid ethyl, cyproheptadine, diphenylpyraline, triprolidine, promethazine, homochlorcyclizine, ammonia, capsaicin, nonylic acid vanillylamide, salicylic acid, methyl salicylate, glycol salicylate, alimemazine, clemastine, mequitazine, dexamethasone, betamethasone, dexamethasone valerate acetate, prednisolone valerate acetate, hydrocortisone butyrate, prednisolone acetate, prednisolone, hydrocortisone acetate, hydrocortisone, cortisone acetate, clobetasone butyrate, triamcinolone acetonide, crotamiton, thymol, eugenol, menthol, camphor, hinokitiol, polyoxyethylene lauryl ether, comfrey extract, Perilla frutescens var. crispa (shiso) extract, sage extract, moutan bark extract, Tila miqueliana extract, and pharmacologically (pharmaceutically) or physiologically acceptable salts thereof.

Further, the nerve extension inhibitor composition or dermal hyperesthesia inhibitor composition of the present invention may optionally contain a single type or 2 or more types of different components that are incorporated in drugs, quasi-drugs or cosmetics, as long as it does not inhibit the effect of the present invention. Such components include those listed in (a) to (g) below.

(a) Anti-inflammatory agent: glycyrrhizic acid, glycyrrhizic acid derivatives, such as dipotassium glycyrrhizinate, monoammonium glycyrrhizinate, glycyrrhetic acid or derivatives thereof, allantoin or derivatives thereof, indomethacin, ibuprofen, ibuprofenpiconol, bufexamac, butyl fulfenamate, bendazac, piroxicam, ketoprofen, felbinac, salicylic acid derivatives, such as methyl salicylate or glycol salicylate.

(b) Vitamins: vitamin A such as retinol, provitamin A such as β-carotene or lycopene, vitamin E such as tocopherol, vitamin B2 such as riboflavin or flavin mononucleotide, vitamin C such as ascorbic acid or dehydroascorbic acid, vitamin D such as ergocalciferol or cholecalciferol, vitamin K such as phylloquinone, vitamin B1 such as y-oryzanol or thiamine, vitamin B6 such as pyridoxine or pyridoxal, vitamin B 12 such as cyanocobalamin, folic acids such as folic acid or pteroylglutamic acid, vitamin B3 such as nicotinic acid or nicotinamide, pantothenic acids such as pantothenic acid or coenzyme A.

(c) Antibacterial agent: isopropylmethylphenol, chlorhexidine hydrochloride, chlorhexidine gluconate, benzalkonium chloride, cetylpyridinium chloride, polyhexamethylene biguanide, triclosan, trichlorocarbanilide, cresol, piroctone olamine, etc.

(d) Antifungal agent: itraconazole, amorolfine hydrochloride, croconazole hydrochloride, terbinafine hydrochloride, neticonazole hydrochloride, butenafine hydrochloride, clotrimazole, ketoconazole, ciclopirox olamine, isoconazole nitrate, econazole nitrate, oxiconazole nitrate, sulconazole nitrate, bifonazole, pimaricin, fluconazole, flucytosine, miconazole, lanoconazole, etc.

(e) Humectant: glycerin, ethylene glycol, propylene glycol, polyethylene glycol, diglycerine trehalose, heparin analogous substance, sodium chondroitin sulfate, collagen, elastin, chitin, chitosan, glycine, aspartic acid, sodium lactate, urea, sodium pyrrolidonecarboxylate, ceramide, cholesterol, phospholipid, chamomile extract, aloe extract, hamamelis extract, rosemary extract, thyme extract, tea extract, Perilla frutescens var. crispa (shiso) extract, etc.

(f) Whitening agent: vitamins, such as the above mentioned vitamin A, vitamin C or vitamin E and pantothenic acid, as well as placenta, arbutin, kojic acid, cysteine, phytic acid, iris, almond, aloe, ginkgophyta, oolong tea, rose fruit, scutellaria root, coptis, hypericaceae, deadnettle, sea weed, pueraria root, gardenia, sophora root, flour, rice germ, rice bran, Perilla frutescens var. crispa (shiso), peony root, cnidii rhizome, mulberry bark, soy bean, tea, angelica acutiloba, carthamus tinctorius, moutan bark, coicis semen, hackberry, etc.

(g) Other examples include various astringents (citric acid, zinc sulfate, sea weed extract, etc.), antioxidants (dibutylhydroxy toluene, sodium edetate, sodium sulfite), anti wrinkle agent (acylated glucosamine, kinetin, hyaluronic acid).

<Components for Formulation>

The nerve extension inhibitor composition or dermal hyperesthesia inhibitor composition of the present invention may further contain a base, a surfactant, a thickener, a preservative, a pH adjustor, a stabilizer, a stimulation reducing agent, an antiseptic agent, a coloring agent, a dispersing agent, a fragrance, etc., as necessary for pharmaceutical formulation and as long as it does not inhibit the effect of the present invention.

Examples of bases include liquid paraffin, paraffin, Vaseline, microcrystalline wax, tulc, myristic acid, lauryl alcohol, cetyl alcohol, cetyl octanoate, isopropyl palmitate, dipentaerythritol fatty acid ester, trimyristic acid glyceryl, methylpolysiloxane, cross-linked polyether modified silicone, cross-linked alkyl modified silicone, etc.

Examples of surfactants include sorbitan fatty acid esters, glycerine fatty acid esters, polyglycerine fatty acid esters, propylene glycol fatty acid esters, hydrogenated castor oil derivatives, polyoxyethylene sorbitan fatty acid esters, glycerin alkyl ethers, etc.

Examples of thickeners include guar gum, carageenan, xanthan gum, dextran, methyl cellulose, hydroxyl ethyl cellulose, hydroxypropyl cellulose, sodium alginate, propylene glycol alginic acid ester, polyvinyl alcohol, polyvinyl pyrrolidone, polyvinyl methyl ether, carboxyvinyl polymer, sodium polyacrylate, polyethylene glycol, bentonite, dextrin fatty acid ester, etc.

Examples of preservatives include benzoic acid, sodium benzoate, dehydroacetic acid, butyl parahydroxybenzoate, ethyl parahydroxybenzoate, methyl parahydroxybenzoate, phenoxy ethanol, etc.

Examples of pH adjustors include inorganic acids (hydrochloric acid, sulfuric acid, phosphoric acid, polyphosphoric acid, boric acid, etc.), organic acids (lactic acid, acetic acid, citric acid, tartaric acid, succinic acid, oxalic acid, etc.), gluconolactone, ammonium acetate, inorganic bases (sodium hydrogen carbonate, sodium carbonate, potassium hydroxide, sodium hydroxide, etc.), organic bases (monoethanolamine, triethanolamine, diisopropanol amine, lysine, etc.).

[Method for Treating or Preventing Dermal Hyperesthesia]

The nerve extension inhibitor or dermal hyperesthesia inhibitor of the present invention, or nerve extension inhibitor composition or dermal hyperesthesia inhibitor composition can be used to carry out a therapeutic method or preventative method of dermal hyperesthesia.

[Therapeutic/Preventative Method of Dermal Hyperesthesia]

A therapeutic/preventative method of dermal hyperesthesia that uses a nerve extension inhibitor or a dermal hyperesthesia inhibitor that is at least one type selected from (1) to (6) below, or a nerve extension inhibitor composition containing said nerve extension inhibitor or a dermal hyperesthesia inhibitor composition containing said dermal hyperesthesia inhibitor as a therapeutic agent/preventative agent, and treats or prevents dermal hyperesthesia symptoms.

(1) Apigenin, luteolin, diosmetin, genkwanin, poncirin, eriodictyol, sterubin, prunetin, which belong to flavonoids, or glycosides or pharmaceutically acceptable derivatives thereof;

(2) Tannin or glycosides or pharmaceutically acceptable derivatives thereof;

(3) A chlorogenic acid or glycosides or pharmaceutically acceptable derivatives thereof;

(4) Stilbenoids encompassing at least resveratrol or glycosides or pharmaceutically acceptable derivatives thereof;

(5) A Prunus jamasakura bark extract, a Polygala root extract, a Hoelen extract, a Glechoma hederacea extract, or an Atractylodis lanceae rhizoma extract, which are crude drug extracts; and

(6) Walnut polyphenol.

[Embodiments of the Therapeutic/Preventative Method of Dermal Hyperesthesia]

Of the therapeutic/preventative agent used in the therapeutic/preventative method of dermal hyperesthesia of the present invention, the nerve extension inhibitor composition and the dermal hyperesthesia inhibitor composition are those described in the above “Nerve Extension Inhibitor Composition, Dermal Hyperesthesia Inhibitor Composition” section.

A dermal external preparation is given as a preferable example of the form for applying a therapeutic/preventative agent, but an oral medicine or an injection is also preferably used. The dosage and method of applying a therapeutic/preventative agent is appropriately selected according to the symptom of dermal hyperesthesia, and it is not particularly limited, but normally, it is used a few times a day at an appropriate amount. The subject for applying a therapeutic/preventative agent is not limited to a human, and animals, particularly mammals are preferable subjects.

[Dermal Hyperesthesia Detection Marker, Dermal Hyperesthesia Diagnostic Method]

<Dermal Hyperesthesia Detection Marker>

As mentioned as the effect of Invention 9, at least one of active HDC and inactive HDC in the skin keratinocyte sample can be used as a detection marker for dermal hyperesthesia. In other words, the HDC expression amount or the HDC activation induction ratio in the skin keratinocytes, as well as the state of nerve extension into the epidermis, may be grasped when the amount of inactive HDC (A) in a specific skin keratinocyte sample, the amount of active HDC (B), or the ratio of (A) and (B) are measured. Therefore, it is possible to detect that the dermal hyperesthesia symptom is expressed, or about to be expressed.

The skin keratinocyte sample of human can be collected, for example, from a test subject by skin biopsy. The inactive HDC amount or the active HDC amount in the collected skin keratinocyte sample can be measured using the Western blot analysis.

<Dermal Hyperesthesia Diagnostic Method>

Measuring the amount of the dermal hyperesthesia detection marker in the skin keratinocyte sample collected from the patient makes it possible to diagnosis that the dermal hyperesthesia symptom is expressed, or to predict that the dermal hyperesthesia symptom is about to be expressed.

### EXAMPLES

Next, the Examples of the present invention are described. The technical scope of the present invention is not limited by the following Examples.

### Sample Experiment 1

### Mouse Scratch Bouts and Histamine Amount in Mouse Skin

### Sample Experiment 1-1

The present Sample Experiment was conducted using ICR mice, and an anionic surfactant, namely, sodium dodecyl sulfate (SDS), was used as a stimulating substance to analyze the scratch bouts of mice. After SDS had been applied, terfenadine (TRF) was administered as an antihistamine to determine its effect on scratch bouts.

Experiment Animal:

Male ICR mice at ages of 7 to 8 weeks, purchased from Japan SLC, were used. The mice were given solid feed under free water supply and subjected to preliminary rearing for 7 days or longer under a constant temperature/constant humidity environment with a temperature of 22±2° C., and a humidity of 55±10% before they were used in the test. The mice for use were used in the test at least 3 days after shearing/depilating the rostral back of each mouse.

Stimulating Substance:

A 10% aqueous solution composed of SDS dissolved in distilled water was applied a single time at 50 μL to the sheared/depilated rostral back of the mice as a stimulating substance against the skin. If the surfactant solution had turned hard before application, it was heated and dissolved in a hot water bath of 37° C. before use. Distilled water was applied to the solvent control group (Vehicle).

Observation of Scratch Bouts:

The scratch bouts of mice were performed in view of the Kuraishi et al. report. That is, a single mouse was put in each chamber of the 4 divided sections in an acrylic box (13×9×35 cm) to be acclimated for at least an hour, and they were video recorded in an environment without any person for at least an hour a day from the day of applying the above 10% SDS aqueous solution (Day 0) to the 4^{th} day after application (Day 4) to record their movement. Then, by watching the video, the number of scratch bouts against the rostral back by the hind leg was counted. A series of scratching movements (movements that include scratching the rostal back, where the solution is applied, with the hind leg, then dropping the hind leg to the ground) were counted as 1 count, and the number of scratch bouts was counted for 60 mins. by visual observation.

Statistical Processing:

Data was shown by average±standard error (SEM). Dunnett's multiple comparisons or Student's t-test was used for statistics. A probability level (p) of below 5% was considered a significant difference. The software used for statistical analysis is StatLight (Yukms Co. Ltd., Tokyo, Japan).

Mouse Scratch Bouts:

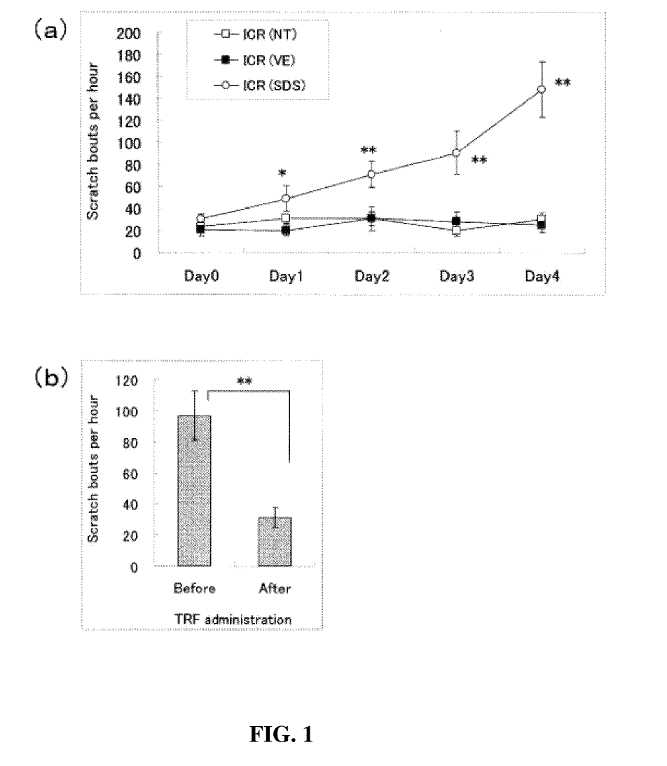

The result of the above observation of scratch bouts is shown in FIG. 1(a). In FIG. 1(a), the vertical axis shows the number of scratch bouts in 60 mins. The plot of ICR (NT) shown by “□” shows an untreated group (a group whose rostal back is sheared/depilated without having anything applied), and the plot of ICR (VE) shown by “▪” shows a solvent control group (Vehicle), and the plot of ICR (SDS) shown by “o” shows a 10% SDS aqueous solution application group. As shown by the result of FIG. 1(a), the scratch bouts of mice do not increase in the untreated group or the solvent control group, but increased significantly over time in the 10% SDS aqueous solution application group.

Meanwhile, ICR mice of the 10% SDS aqueous solution application group mentioned above were taken out of the observation room at Day 3, then left to rest for 3 hrs. under a water supplied/feed supplied environment, after which they were put in the observation room and acclimated for 1 hr. Then, TRF was administered 30 mins. before the observation of the scratch bouts by video recording discussed later. TRF was dissolved in 0.5% carboxymethyl cellulose (CMC-Na), and orally administered at a ratio of 0.05 mL (30 mg/kg) per 10 g of body weight. Then, after 30 mins. had passed from the oral administration of TRF, the scratch bouts of the ICR mice were video recorded as described in the above “Observation of Scratch Bouts.” The reason the mice were “left to rest for 3 hrs. before observation” was that the amount of each mouse's movement was expected to decrease by a continuous observation of movements, so the mice were given temporarily rest under a water supplied/feed supplied environment. The observation result of scratch bouts is shown in the “After” bar graph of FIG. 1(b). The vertical axis of FIG. 1(b) shows the number of scratch bouts in 60 mins. The “Before” bar graph of FIG. 1(b) shows the number of scratch bouts of a mouse in the 60 mins. immediately before the oral administration of TRF, and this is the same as the number of scratch bouts at Day 3 of the 10% SDS aqueous solution application group in FIG. 1(a). A comparison of “After” and “Before” indicates that the scratch bouts of mice were suppressed by administering TRF.

The assessment of this Sample Experiment is described in the section “Discussion” presented below.

### Sample Experiment 1-2

The present Sample Experiment was conducted using mast cell deficient mice and normal control mice, and SDS was used as a stimulating substance to analyze the scratch bouts of mice. Further, the amount of histamine in the skin of each mouse was assessed after SDS had been applied.

Experiment Animal:

Male mast cell deficient mice at ages of 7 to 8 weeks (WBB6F1-W/W^{V} mouse), purchased from Japan SLC, and normal control mice that are wild mice at ages of 7 to 8 weeks (WBB6F1-+/+mouse) were used. The mice were given solid feed under free water supply and subjected to preliminary rearing for 7 days or longer under a constant temperature/constant humidity environment with a temperature of 22±2° C., and a humidity of 55±10% before they were used in the test. The mice for use were used in the test at least 3 days after shearing/depilating the rostral back of each mouse.

Stimulating Substance:

A 10% aqueous solution composed of SDS dissolved in distilled water was applied a single time at 50 μL as a stimulating substance against the skin to the sheared/depilated rostral back of the mice mentioned above. If the surfactant solution had turned hard before application, it was heated and dissolved in a hot water bath of 37° C. before use. Distilled water was applied to the solvent control group (Vehicle).

Observation of Scratch Bouts:

The observation and statistical processing of the scratch bouts of mice were performed in exactly the same manner as shown in the “Observation of Scratch Bouts” and the “Statistical Processing” of Sample Experiment 1-1.

Scratch Bouts of Mouse:

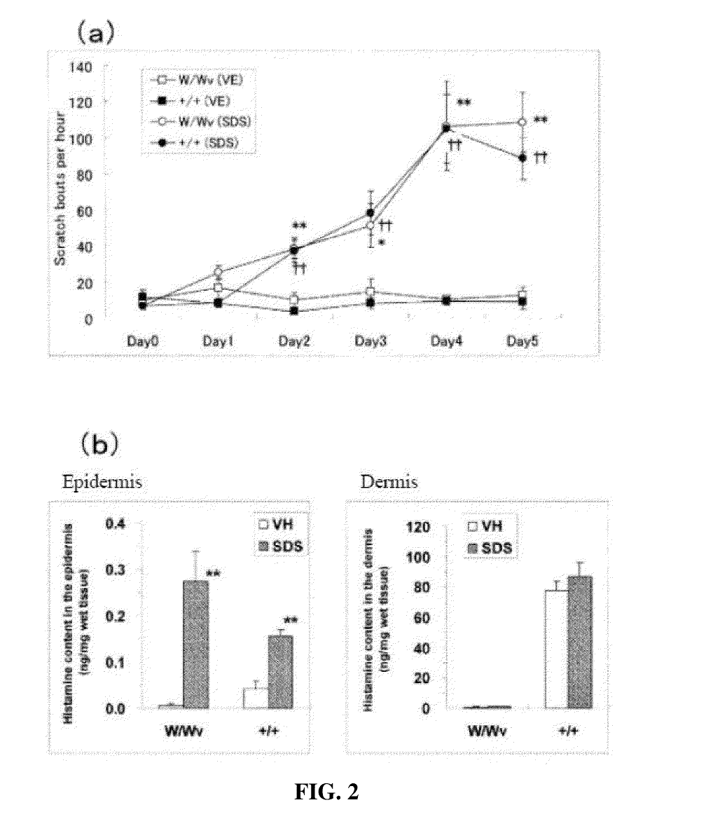

The result for the above observation of scratch bouts is shown in FIG. 2(a). In FIG. 2(a), the vertical axis shows the number of scratch bouts in 60 mins. The plot of W/Wv (VE) shown by “□” shows the solvent control group (Vehicle) of mast cell deficient mice; the plot of +/+(VE) shown by “▪” shows the solvent control group (Vehicle) of normal control mice; the plot of W/Wv (SDS) shown by “o” shows a 10% SDS aqueous solution application group of mast cell deficient mice; and the plot of +/+(SDS) shown by “” shows a 10% SDS aqueous solution application group of normal control mice. As shown by the result of FIG. 2(a), the scratch bouts of mice increased significantly with time for both the mast cell deficient mice and the normal control mice in the 10% SDS aqueous solution application group, and no significant difference was seen between both mice, since their scratch bouts increased by a same degree.

Amount of Histamine in Mouse Skin:

Meanwhile, skin was collected from each of the mast cell deficient mice and normal control mice in the 10% SDS aqueous solution application group mentioned above at Day 4 while the mice were under anesthesia after perfusion with a phosphate buffer solution (PBS) using the cardiac blood circulation system (punched out with a biopsy punch in a circular shape of a diameter of 18 mm). The amounts of histamine in the collected skins were measured.

In other words, each collected skin was immersed in PBS of 60° C. for 30 secs., then separated into the epidermis and the dermis. Subsequently, the amounts of histamine in the epidermis and the dermis were measured using the measurement kit (histamine enzyme immunoassay kit (Immunotech, Marseilles, France)). To measure the amount of histamine, tissues of both the epidermis and the dermis were pulverized to create tissue suspensions, which were subjected to centrifugation, then supernatants were collected to measure the amounts of histamine in the supernatants.

(1) Amount of Histamine in Mouse Epidermis:

An assessment was made of whether the SDS treatment led to an increase in the level of histamine in the mouse epidermis. The result is shown in the left graph of FIG. 2(b). The vertical axis of the left graph of FIG. 2(b) shows the amount of histamine in the mouse epidermis. In addition, “W/Wv” on the horizontal axis shows that the result is for mast cell deficient mice, and “+/+” shows that the result is for normal control mice. Also, the white bar graph (left) shows a SDS applied group, and the gray bar graph (right) shows a solvent control group (Vehicle). As seen from the graph, the amounts of histamine in the epidermis of both the mast cell deficient mice and the normal control mice are significantly high in the SDS applied group compared to the solvent control group.

(2) Amount of Histamine in Mouse Dermis:

An assessment was made of whether the SDS treatment led to an increase in the level of histamine in the mouse dermis. The result is shown in the right graph of FIG. 2(b). The vertical axis of the right graph of FIG. 2(b) shows the amount of histamine in the mouse epidermis. Also, the indication of “W/Wv” and “+/+” on the horizontal axis, the white bar graph (left), the gray bar graph (right) respectively mean the same as corresponding items in the left graph of FIG. 2(b). As seen from the graph, the amounts of histamine in the dermis of both the mast cell deficient mice and the normal control mice in the SDS applied group have barely increased compared to the solvent control group.

The assessment of this Sample Experiment will be presented in the “Discussion” section provided below.

### Sample Experiment 1-3

The present Sample Experiment was conducted using ICR mice, and SDS was used as a stimulating substance. After SDS was applied, the amount of histamine in the skin of each mouse was assessed, and so was the HDC expression amount in the skin of each mouse.

Experiment Animal:

The same ICR mice as used in Sample Experiment 1-1 was used. Treatments of the ICR mice, such as preliminary rearing, and the shearing/depilating of the rotal back, were performed similarly to Sample Experiment 1-1.

Stimulating Substance:

A 10% aqueous solution obtained by dissolving SDS in distilled water was applied to the mouse rostal back as a stimulating substance against the skin similarly to Sample Experiment 1-1. Distilled water was applied to the solvent control group (Vehicle).

Amount of Histamine in Mouse Skin:

Skin was collected by operations similar to that mentioned in “Amount of Histamine in Mouse Skin” in Sample Experiment 1-2 from each ICR mouse of the 10% SDS aqueous solution application group and the solvent control group mentioned above after 4 days had passed from application (Day 4). The collected skins were separated into the epidermis and the dermis, and the amounts of histamine in those skins were measured.

(1) Amount of Histamine in Mouse Epidermis:

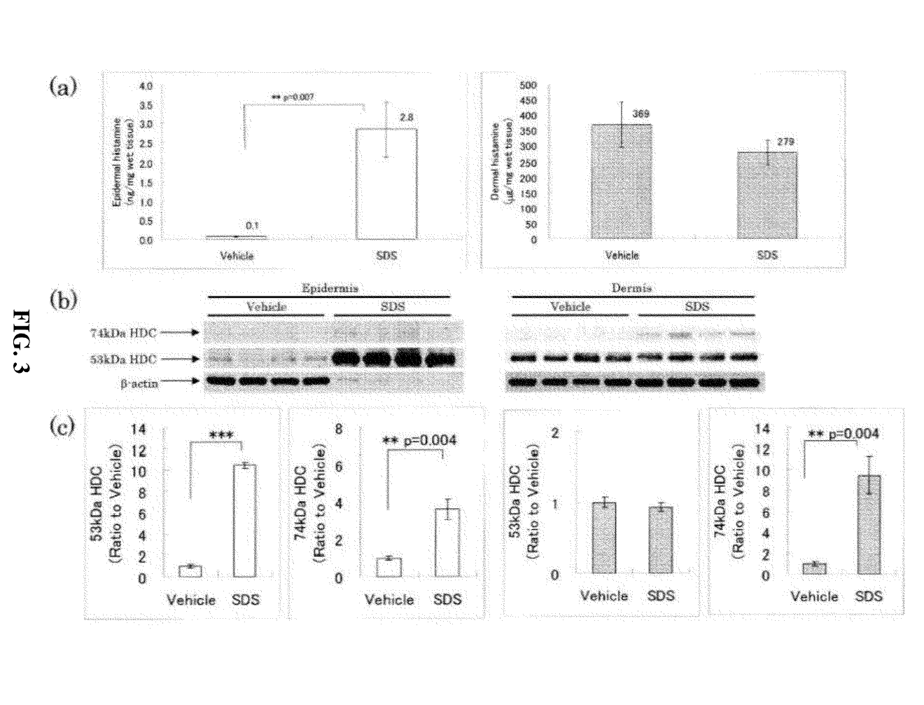

The measurement results concerning the amount of histamine in the mouse epidermis are shown in the left graph of FIG. 3(a). The vertical axis of the graph shows the amount of histamine in the mouse epidermis. In addition, the “Vehicle” and “SDS” on the horizontal axis respectively indicate the amount of histamine in the epidermis of the solvent control group and the amount of histamine in the epidermis of the 10% SDS aqueous solution application group. It can be seen that the amounts of histamine in the epidermis of the SDS application group are significantly higher compared to the solvent control group.

(2) Amount of Histamine in Mouse Dermis:

The measurement results concerning the amount of histamine in the mouse dermis are shown in the right graph of FIG. 3(a). The vertical axis of the graph shows the amount of histamine in the mouse dermis. In addition, the “Vehicle” and “SDS” on the horizontal axis respectively indicate the same matter as those in the left graph of FIG. 3(a). It can be seen that the amount of histamine in the dermis of the 10% SDS aqueous solution application group does not differ significantly from that of the solvent control group.

HDC Expression Amount in Mouse Epidermis and Dermis:

Concerning the epidermis and dermis obtained by the method in the “Amount of Histamine in Mouse Skin” section above, the HDC in the epidermis and the HDC in the dermis were quantitatively determined by the Western blot analysis. The protein in the epidermis and the dermis was extracted using the Mammalian cell lysis kit (Sigma, Tokyo, Japan).

The protein extract was subjected to centrifugation and the obtained supernatant was used as a protein solution. The amount of protein in the protein solution was quantitatively determined using a protein quantification kit, 2-D Quant Kit of GE Health Care Bioscience Co. Then, a sample buffer containing a reducing agent, 2-mercaptoethanol, was added to a certain amount of protein, and the mixture was reacted at 95° C. to cut the disulfide bond in the protein structure. This enables electrophoresis that reflects the molecular amount.

The protein solution processed in the above manner was subjected to the Western blot analysis. In other words, electrophoresis was performed according to the following electrophoresis condition. As a result, protein was separated according to the molecular amount. Then, the separated protein was transcribed to the membrane according to the following transcription condition.

[Electrophoresis Condition]

Applied gel: Nupage 4%-12% Bis-tris gels (Invitrogen Corp., Carlsbad, Calif.)
Electrophoresis buffer: 3-morpholinopropanesulfonic acid (MOPS) buffer
Current/electrophoresis time: 200V, 100 mins.

### [Transcription Condition]

Applied membrane: Polyvinylidene Fluoride (PVDF) membrane
Electrophoresis buffer: NuPAGE transcription buffer (Invitrogen Corp., Carlsbad, Calif.) pos Current/electrophoresis time: 30V, 60 mins.

The membrane was cut at about 50 kDa after transcription. Then, blocking was performed with a 1% skim milk aqueous solution. After blocking, a solution of a primary antibody, namely a rabbit polyclonal anti-L-histidine decarboxylase (HDC) antibody (Progen Biotechnik GmbH, Heidelberg, Germany), diluted 1000 folds with Can Get Signal 1 (Toyobo Co. Ltd., Osaka, Japan) was reacted with the membrane on the high molecular size side, and a solution of a primary antibody, namely a rabbit polyclonal anti-β-actin antibody (Abcam, Tokyo, Japan), diluted 1000 folds with Can Get Signal 1 (Toyobo Co. Ltd., Osaka, Japan) was reacted with the membrane of the low molecular size side at 4° C., overnight. After processing the primary antibody and then washing each membrane with PBS-T (a solution comprising PBS and 0.1% Tween20), each membrane was reacted with a solution of a secondary antibody, fluorophore-labeled donkey anti-rabbit IgG (H+L) antibody (Invitrogen Co. Carlsbad, Calif.), diluted 1000 folds with Can Get Signal 2 (Toyobo Co. Ltd., Osaka, Japan). After processing the secondary antibody and then washing each membrane with PBS-T (a solution comprising PBS and 0.1% Tween20), a band was detected with a fluorescent scanner. Then, the detected band was determined by quantification using an image analysis software Scion Image (Scion. Corp., Frederick, Md., USA). The Scion Image is a software that selects the band of the protein to be quantified, reads the band area and the density of the color tone, and detects the numerical value corresponding to the protein expression amount based on those information.

HDC and β-actin (component of the cytoskeleton) are detected from the above processing. The HDC to be detected are active HDC (53 kDa) and inactive HDC (74 kDa).

The analysis results concerning the HDC expression amount are shown in FIG. 3(b) and FIG. 3(c). The left diagram in FIG. 3(b) shows the result of the Western blot analysis of epidermis, and the right diagram shows the result of the Western blot analysis of dermis. FIG. 3(c) shows graphs that correspond to each band of FIG. 3(b) by converting the band to a numerical value. In the diagram, “Vehicle” and “SDS” respectively refer to the solvent control group and the 10% SDS aqueous solution application group. The numerical values of the 10% SDS aqueous solution application group in FIG. 3(c) are relative values against values of the solvent control group set as 1. In FIG. 3(c), four graphs are shown in parallel, in which the left graph is the measurement result for active HDC (53 kDa) concerning epidermis, the second graph from the left is the measurement result for inactive HDC (74 kDa) concerning epidermis, the third graph from the left is the measurement result for active HDC (53 kDa) concerning dermis, and the right graph is the measurement result for inactive HDC (74 kDa) concerning dermis.

The assessment of this Sample Experiment will be presented in the “Discussion” section provided below.

[Sample Experiment 2: Scratch Bouts of Mouse]

This Sample Experiment is the above Sample Experiment 1 modified mainly by performing the experiment section of the “Scratch Bouts of Mouse” under a different condition.

Experiment Animal:

Male mast cell deficient mice at ages of 7 to 8 weeks (WBB6F1-W/W^{V} mouse), purchased from Japan SLC, and normal control mice that are wild mice at ages of 7 to 8 weeks (WBB6F1-+/+mouse) were used. They were given solid feed under free water supply and subjected to preliminary rearing for 7 days or longer under a constant temperature/constant humidity environment with a temperature of 22±2° C., and a humidity of 55±10% before they were used in the test. The mice for use were subjected to the test at least 3 days after shearing/depilating of the rostral back.

Stimulating Substance:

A 10% aqueous solution obtained by dissolving an anionic surfactant, sodium laurate (SL), in distilled water was applied a single time at 50 μL as a stimulating substance against the skin to the sheared/depilated rostral back of the mouse mentioned above. If the surfactant solution turned hard before application, it was heated and dissolved in a hot water bath of 37° C. before use. Distilled water was applied to the solvent control group.

Observation of Scratch Bouts:

The scratch bouts of the mice were conducted in view of the Kuraishi et al. report. That is, a single mouse was put in each of the 4 divided sections of an acrylic box (13×9×35 cm) to be acclimated for at least 1 hr., and they were video recorded in an environment without any person to record their action. Then, by watching the video, the number of scratch bouts against the rostral back by the hind leg was counted. A series of scratching movements (movements that include scratching the rostal back, where the solution is applied, with the hind leg, then dropping the hind leg to the ground) was counted as 1 count, and the number of times scratching was performed in 60 mins. was counted by visual observation.

Statistical Processing:

Data was shown by average±standard error (SEM). Dunnett's multiple comparisons or Student's t-test was used for statistics. A probability level (p) of below 5% was considered as a significant difference. The software used for statistical analysis is StatLight (Yukms Co. Ltd., Tokyo, Japan).

Scratch Bouts of Mouse:

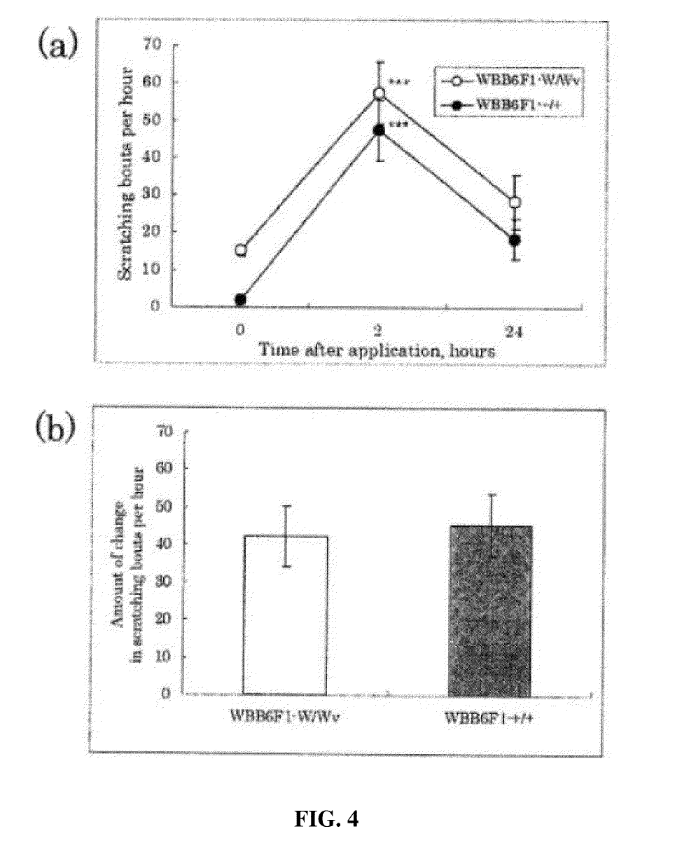

When the SL aqueous solution was applied to the mast cell deficient mice and the wild type mice, constituting normal control mice, the scratch bouts clearly increased 2 hrs. after application (the time of application was set to 0 hr.) for both mice. Further, the number of scratch bouts was about the same for the mast cell deficient mice and the normal control mice as shown in FIG. 4(b).

The assessment of this Sample Experiment will be presented in the “Discussion” section provided below.

[Sample Experiment 3: Amount of Histamine in Mouse Skin]

This Sample Experiment is the above Sample Experiment 1 modified mainly by performing the experiment section of the “Amount of Histamine in Mouse Skin” under a different condition.

Experiment Animal:

Male ICR mice at ages of 7 to 8 weeks, purchased from Japan SLC, were used. They were given solid feed under free water supply and subjected to preliminary rearing for 7 days or longer under a constant temperature/constant humidity environment with a temperature of 22±2° C., and a humidity of 55±10% before they were used in the test. The mice for use were subjected to the test at least 3 days after shearing/depilating of the rostral back.

Stimulating Substance:

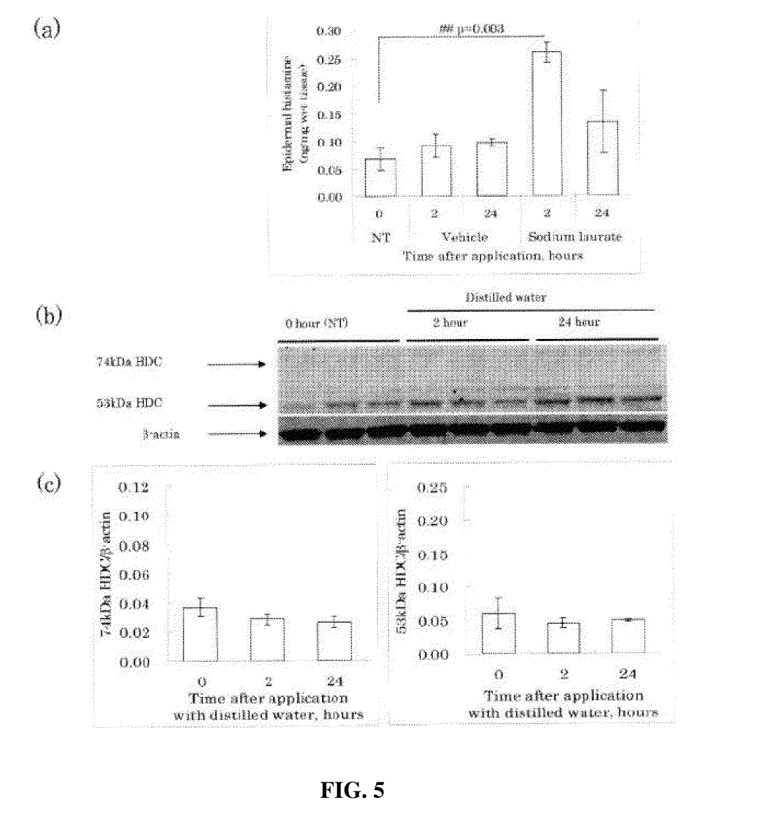

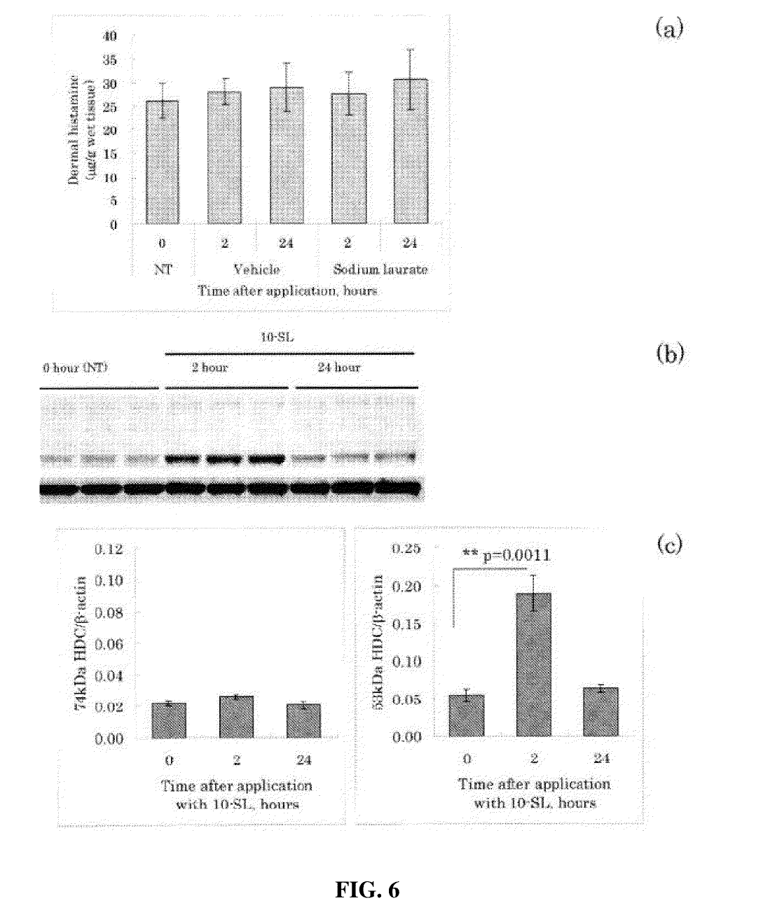

A 10% aqueous solution obtained by dissolving SL in distilled water was applied as a stimulating substance against the skin at 50 μL to the sheared/depilated rostral back of the mice mentioned above. Distilled water was applied to the solvent control group (Vehicle). Note concerning FIG. 5 and FIG. 6 to be described later that the mice that were not treated with the above SL aqueous solution or distilled water are called “NT (not treated).”

Amount of Histamine in Mouse Skin:

The amount of histamine in skin was measured before applying a surfactant, 2 hrs. after application, and 24 hrs. after application (the time of application was set to 0 hr.). Skins were collected while the mice were under anesthesia after perfusion with a phosphate buffer solution (PBS) using the cardiac blood circulation system (punched out with a biopsy punch in a circular shape of a diameter of 18 mm). The amounts of histamine in the collected skins were measured.

After the collected skins were immersed in PBS at 60° C. for 30 secs., they were separated into the epidermis and the dermis. Then, the amounts of histamine in the epidermis and the dermis were measured using the measurement kit. To measure the amount of histamine, tissues of both the epidermis and the dermis were pulverized to create tissue suspensions, which were subjected to centrifugation, then supernatants were collected to measure the amounts of histamine in the supernatants.

Amount of Histamine in ICR Mouse Epidermis and Dermis:

(1) An assessment was made of whether the SL treatment led to an increase in the level of histamine and to the activation of HDC in the mouse epidermis. The result was that the active HDC (53 kDa) increased significantly in the mouse epidermis 2 hrs. after the SL aqueous solution was applied and returned to the pre-application level 24 hrs. after application as shown in FIG. 5(b) and (c). Further, the histamine level in the epidermis also increased 2 hrs. after application as shown in FIG. 5(a) and returned to the pre-application level 24 hrs. after application.

(2) On the other hand, the histamine level of the dermis, assessed similarly as the epidermis, was within the range of measurement error at any observation timing (before application, 2 hrs. after application, and 24 hrs. after application), and no substantial change was observed.

The assessment of this Sample Experiment will be presented in the “Discussion” section provided below.

### Example 1

### Measurement of Nerve Extension into Epidermis

Male ICR mice at ages of 7 to 8 weeks were used as experiment animals. The rostral backs of the mice were sheared 6 cm² (233 3 cm) at least 3 days before the test.

Each of the following substances were used as the subject substance to perform the Examples below: tannic acid, resveratrol, apigenin, luteolin, sterubin, chlorogenic acid, diosmetin, poncirin, eriodictyol, prunetin, walnut polyphenol, Prunus jamasakura bark extract, Poly gala root extract, Hoelen root extract, Glechoma hederacea extract and Atractylodis lanceae rhizoma extract.

The mice were divided into the subject substance application group (n=3−4) and the solvent control group (n=3−4), and a 10% aqueous solution of a stimulating substance, sodium dodecyl sulfate (SDS), was applied to the rostal back of each group once per day at 50 μl for 5 consecutive days to induce nerve extension into the epidermis. Three hours after the SDS aqueous solution was applied, a solution of the subject substance adjusted with 50% ethanol to achieve 2.0 mass % was applied to the subject substance application group, and 50% ethanol was applied to the solvent control group, each applied to the rostal back at 50 μl to assess the nerve extension suppression effect of the subject substance.

On the fifth day of the test, the skin of each mouse was cut off after perfusion with a phosphate buffer (0.1 M phosphate-buffered saline; PBS). The cut off skin was immersed and fixed for 24 hrs. with a 4% paraformaldehyde solution. Then, after the solution was replaced with a 30% sucrose aqueous solution, a block of a frozen skin sample was prepared using an embedding agent for freezing (OTC compound; Tissue Tek; Sakura, Tokyo, Japan). A thin section (thickness, 40 μm) was cut out from the frozen block with a cryostat. Then, the skin section was reacted with a primary antibody, namely, a solution of the rabbit polyclonal anti-protein gene product 9.5 (PGP 9.5) antibody (Ultraclone, Isle of Wight, UK) diluted 1000 folds with PBS-T (a solution comprising PBS and 0.1% Tween20) containing 1.5% FBS, at 4° C. overnight. Then, a reaction was performed with a secondary antibody, namely, a solution of fluorophore-labeled donkey anti-rabbit IgG (H+L) antibody (Invitrogen Co.) diluted 1000 folds with PBS-T (a solution comprising PBS and 0.1% Tween20) containing 1.5% FBS, for 2 hrs. at room temperature. The skin sample that was immunostained was observed with a fluorescence phase-contrast microscope.

The obtained section image is an image of an immunostained nerve in the mouse's skin as shown in FIG. 7, and the white section in the epidermis and dermis in FIG. 7 shows the nerve.

However, in FIG. 7, white lines are manually drawn to indicate the surface section of the epidermis as well as the boundary of the epidermis and the dermis for descriptive purposes. Such section images were assessed using an image analysis software (Image J software (NIH, Bethesda, Md., USA)).

The assessment of this Example is described in the section “Discussion” presented below.

### Example 2

### Data Analysis

The assessment using the image analysis software was performed specifically by converting the signal strength (fluorescent strength) of the nerve in the epidermis to numerical values, and the obtained numerical values were arranged in graphs. That is, an epidermis part in a skin section image like that of FIG. 7 was selected. Then, the signals in the epidermis were separated into a part equal to or higher than the previously set threshold value (white spot or line showing the nerve) and the part below the previously set threshold value (gray-black). The signal strength of the part equal to or higher than the previously set threshold value was converted to a numerical value using the image analysis software. The numerical value of the signal strength was obtained in this manner for each group.

Note that a water application group (n=3−4) was prepared in addition to the subject substance application group and the solvent control group. In this group, the application of the 10% aqueous solution of SDS in Example 1 was replaced with application of water, and subsequent operations, namely the application of a 50% ethanol solution of the subject substance in the subject substance application group and the application of 50% ethanol in the solvent control group, were omitted, but all other operations were performed in a manner completely identical to Example 1.

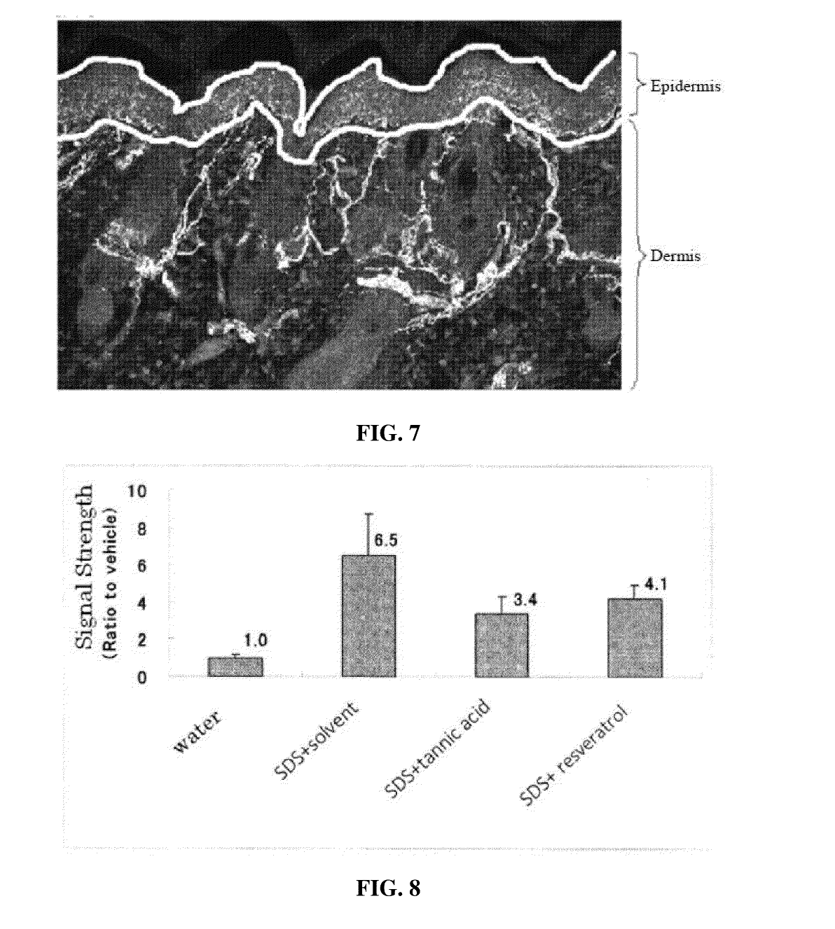

Then, the signal strength obtained by using the above image analysis software for the section image from the water application group was determined as the reference value “1” to obtain the relative values of the signal strength of the subject substance application group and the solvent control group. The results for the above subject substances are shown in FIG. 8 to FIG. 11.

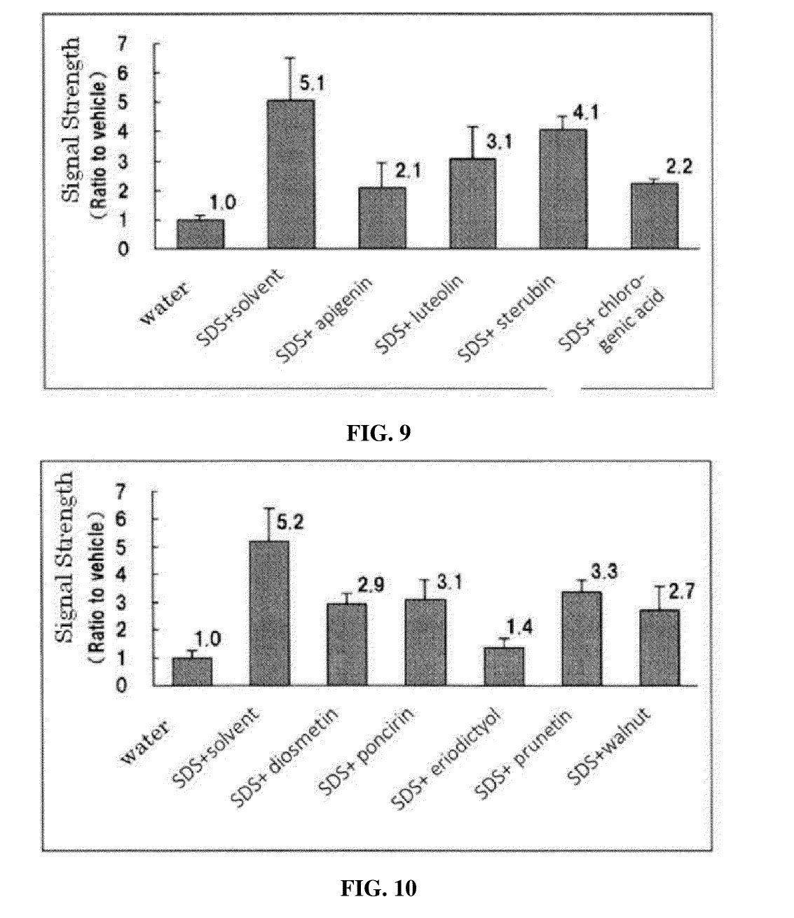

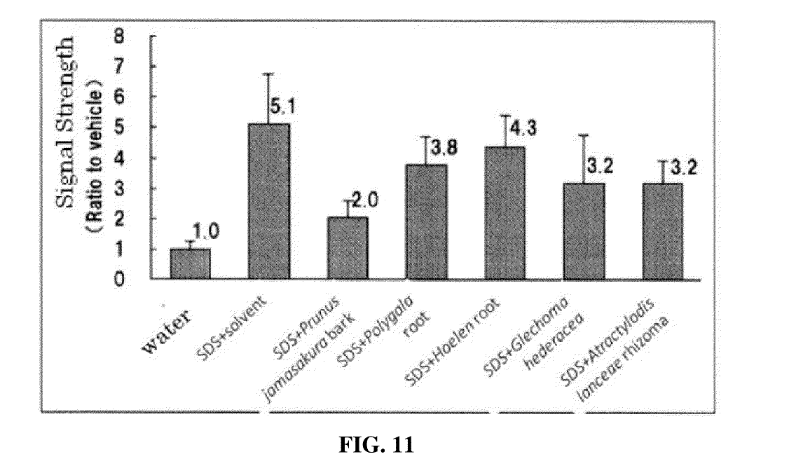

In FIG. 8 to FIG. 11, a graph marked as “water” represents the water application group and a graph marked as “SDS+solvent” represents the solvent control group. For example, a graph marked as “SDS+tannic acid” represents a subject substance application group using that subject substance. In FIG. 10 and FIG. 11, “walnut” refers to walnut polyphenol, and “Prunus jamasakura bark,” “Polygala root,” “Hoelen root extract,” “Glechoma hederacea” and “Atractylodis lanceae rhizoma” respectively refer to Prunus jamasakura bark extract, Polygala root extract, Hoelen root extract, Glechoma hederacea extract and Atractylodis lanceae rhizoma extract. The numerical value added above each bar graph is the height of the bar graph, that is, the relative value of the signal strength.

The assessment of this Example is described in the section “Discussion” presented below.

[Discussion on Sample Experiments and Examples]

(1) According to Sample Experiment 1-1, the scratch bouts of mice increase significantly by applying an SDS aqueous solution and the scratch bouts are effectively suppressed by oral administration of an anti-histamine drug, TRF, as shown by FIGS. 1(a) and (b), so these reactions are histamine dependent. And as mentioned above, it is generally understood that the histamine is emitted from mast cells by degranulation. Hence, the validity of such understanding was assessed using mast cell deficient mice in Sample Experiment 1-2.

According to Sample Experiment 1-2, the scratch bouts of mice increased significantly for both the mast cell deficient mice and the normal control mice in the 10% SDS aqueous solution application group by applying an SDS aqueous solution, and the amount of histamine in the epidermis increased significantly compared to the solvent control group, but the amount of histamine in the dermis did not increase from the solvent control group, as shown in FIGS. 2(a) and (b). Additionally, the amount of histamine in the dermis of the mast cell deficient mice was obviously lower than the normal control mice. In other words, histamine in the dermis has little connection with SDS-induced scratch bouts.

In summary, it can be understood that the mast cell derived histamine does not affect the scratch bouts of mice (movements relating to itch) induced by an SDS aqueous solution application, but the histamine derived from keratinocytes in the epidermis controls the reaction.

According to Sample Experiment 1-3, the amount of histamine in the epidermis increased significantly by applying SDS, but the amount of histamine in the dermis showed almost no change, as shown by FIG. 3(a).

In addition, as shown in FIG. 3(b) and FIG. 3(c), the 53 kDa-HDC expression amount and the 74 kDa-HDC expression amount increased by applying SDS. On the other hand, the 74 kDa-HDC expression amount in the dermis increased significantly but the 53 kDa-HDC expression amount did not change. Accordingly, it is understood that the SDS aqueous solution application caused an increase in the HDC (particularly 53 kDa-HDC) expression amount in the epidermis, and the histamine amount increased as the result. This amount of histamine in the epidermis is considered as the cause of scratch bouts.

(2) According to Sample Experiment 2, the mast cell deficient mice and the normal wild type mice, constituting the control, showed the same level of scratch bouts when the stimulating substance was applied to the skin, as seen from FIG. 4. In other words, the mast cell deficient mice experienced the same itch as the normal mice. On the other hand, Examples 1 and 2 show that application of a similar stimulating substance to normal ICR mice induces nerve extension into the epidermis. This fact suggests that model mice whose mast cells are deficient may also experience nerve extension into the epidermis.

(3) According to Sample Experiment 3, application of a stimulating substance to the skin of ICR mice increases the active HDC in the epidermis, but not in the dermis, as seen from FIGS. 5, 6. This active HDC is considered to be an inactive HDC that had been activated by the work of the stimulating substance in the keratinocytes in the epidermis. Hence, at least one of the active HDC and the inactive HDC in the skin keratinocyte sample can be used effectively as a marker for detecting dermal hyperesthesia.

(4) According to Examples 1 and 2, the nerve extension into the epidermis due to application of a stimulating substance to the skin of ICR mice is suppressed to a great extent for the above subject substances, which are nerve extension inhibitors or dermal hyperesthesia inhibitors of the present invention, compared to the solvent control group as seen from FIG. 8 to FIG. 11. From the above Sample Experiment 1 and Sample Experiment 2, it can be seen that such nerve extension into the epidermis is caused by histamine derived from HDC activation in the keratinocytes in the epidermis.

### INDUSTRIAL APPLICABILITY

The present invention provides a nerve extension inhibitor, dermal hyperesthesia inhibitor, etc., that effectively suppress nerve extension and dermal hyperesthesia based on the newly found nerve extension mechanism.

1. A nerve extension inhibitor that is one or more types selected from (1) to (6) below:
(1) Apigenin, luteolin, diosmetin, genkwanin, poncirin, eriodictyol, sterubin, prunetin, which belong to flavonoids, or glycosides or pharmaceutically acceptable derivatives thereof;
(2) Tannin or glycosides or pharmaceutically acceptable derivatives thereof;
(3) A chlorogenic acid or glycosides or pharmaceutically acceptable derivatives thereof;
(4) Stilbenoids encompassing at least resveratrol or glycosides or pharmaceutically acceptable derivatives thereof;
(5) A Prunus jamasakura bark extract, a Polygala root extract, a Hoelen extract, a Glechoma hederacea extract, or an Atractylodis lanceae rhizoma extract, which are crude drug extracts; and
(6) Walnut polyphenol.

2. A nerve extension inhibitor composition containing the nerve extension inhibitor according to claim 1.

3. The nerve extension inhibitor composition according to claim 2, wherein the nerve extension inhibitor composition is a drug, a quasi-drug or cosmetics.

4. The nerve extension inhibitor composition according to claim 2, wherein the nerve extension inhibitor composition is an external preparation for skin.

5. A dermal hyperesthesia inhibitor that is one or more types selected from (1) to (6) below:
(1) Apigenin, luteolin, diosmetin, genkwanin, poncirin, eriodictyol, sterubin, prunetin, which belong to flavonoids, or glycosides or pharmaceutically acceptable derivatives thereof;
(2) Tannin or glycosides or pharmaceutically acceptable derivatives thereof;
(3) A chlorogenic acid or glycosides or pharmaceutically acceptable derivatives thereof;
(4) Stilbenoids encompassing at least resveratrol or glycosides or pharmaceutically acceptable derivatives thereof;
(5) A Prunus jamasakura bark extract, a Polygala root extract, a Hoelen extract, a Glechoma hederacea extract, or an Atractylodis lanceae rhizoma extract, which are crude drug extracts; and
(6) Walnut polyphenol.

6. A dermal hyperesthesia inhibitor composition containing a dermal hyperesthesia inhibitor according to claim 5.

7. The dermal hyperesthesia inhibitor composition according to claim 6, wherein the dermal hyperesthesia inhibitor composition is a drug, a quasi-drug or cosmetics.

8. The dermal hyperesthesia inhibitor composition according to claim 6, wherein the dermal hyperesthesia inhibitor composition is an external preparation for skin.

9. A dermal hyperesthesia detection marker that is at least one of an active HDC or an inactive HDC in a skin keratinocyte sample.

10. The nerve extension inhibitor composition according to claim 3, wherein the nerve extension inhibitor composition is an external preparation for skin.

11. The dermal hyperesthesia inhibitor composition according to claim 7, wherein the dermal hyperesthesia inhibitor composition is a external preparation for skin.
